(19) United States
(12) Patent Application Publication
(10) Pub. No.: US 2018/0252785 A1
(43) Pub. Date: Sep. 6, 2018

(54) HYBRID TOF-PET/MRI TRANSCEIVER COIL

(71) Applicant: UNIWERSYTET JAGIELLONSKI, KRAKOW (PL)

(72) Inventors: BARTOSZ GLOWACZ, Zory (PL); MARCIN ZIELINSKI, Krakow (PL); PAWEL MOSKAL, Czulowek (PL)

(21) Appl. No.: 15/754,399

(22) PCT Filed: Sep. 3, 2015
(86) PCT No.: PCT/PL2015/050035

§ 371 Date: Feb. 22, 2018

(51) Int. Cl. G01R 33/48; A61B 6/03; A61B 6/00; G01R 33/34; G01T 1/16; A61B 5/055

(52) U.S. Cl. CPC G01R 33/481; A61B 6/037; A61B 5/055; G01R 33/34076; G01T 1/1603; A61B 6/4417

(57) ABSTRACT

The present invention relates to the dedicated hybrid TOF-PET/MRI transceiver coil, comprising: MRI coils in a form of complex shape wire antennas, electronic circuit module allowing for transmitting MRI radiofrequency pulse, and receiving response in a form of magnetic resonance signal, PET detectors arranged longitudinally, fixed permanently to the MRI coils system mechanical support, an electronic signal processing unit, wherein the PET detectors are in a form of plastic scintillating strip modules equipped with photoelectric converter units at both ends to convert a light signals from the scintillating module to electrical signals and composed of non-magnetic materials.

The present invention relates to the dedicated transmit receive coil (transceiver coil) for medical imaging with Magnetic Resonance Imaging technique (MRI) integrated with a system for medical imaging with Positron Emission Tomography technique (PET) with an ability of time-of-flight (TOF) measurement (so called TOF-PET).

Nowadays devices based on the detection of nuclear and electromagnetic radiation are in common use for the diagnosis of the interior of the human body. The most technologically advanced are positron emission tomography (PET), computed tomography (CT) and magnetic resonance imaging (MRI) scanners. PET delivers metabolic images of chosen substances administered to the body, while CT and MRI provides anatomical and morphological images, respectively. In order to enhance diagnostic capabilities the PET scanners are often combined with CT or MRI devices. Performing both metabolic and anatomical or metabolic and morphological images gives not only advantage of increasing information available for the physician but it also permits to improve the quality of the PET images enabling their precise corrections for the radiation attenuation inside the examined object.

PET/CT modalities are presently commonly used in hospitals, whereas PET/MRI imaging systems are still at the early stage of implementation in medical practice. The state of the art of the present development of PET-MRI devices is described e.g. in the recent topical review by S. Vandenberghe, P. K. Marsden, “PET-MRI: a review of challenges and solutions in the development of integrated multimodality imaging”, Physics in Medicine and Biology 60 (2015) R115-R154.

There are several commercial realizations of the PET-MRI concept, however, these solutions are only capable to work in the sequential diagnostic mode—PET and MRI imaging techniques are performed one after another.

In US 20070102641 A1 a solution regarding the combined PET/MRI device has been disclosed where the PET detector have been placed at the periphery of a MRI scanner part. This solution allows different modes of work, namely sequential and simultaneous PET and MRI. However, in such a configuration of PET detectors, one has to take into account restrictions when local transmit-receiver MRI coils are used. Transceiver coils are well known and commonly used in MRI imaging of body parts such as head, chest and limbs, improving the image quality with respect to the use of the whole-body coil built in the MRI system, as the local coils are much closer to the object being imaged, what increases the coil filling factor—parameter on which the registered MRI signal depends. The use of such a devices is possible, however the coils are made of a plastic parts and metal conductors which are on the way of gamma quanta flying from the positron and electron annihilation point in the diagnosed volume. This fact could cause the worsening of a spatial resolution of the PET diagnostic images, as the field of view for the PET detectors is limited.

Patent WO 2015028603 A1 discloses a hybrid TOF-PET/MRI tomograph comprising a TOF-PET tomograph and an MRI tomograph, wherein the TOF-PET tomograph comprises polymer scintillation strips arranged circumferentially inside the working area of the magnetic field of the receiving-transmitting coil of the MRI tomograph and photoelectric converters for converting light signals from the scintillation strips to electrical signals, wherein the photoelectric converters are arranged outside the working area of magnetic field of the MRI tomograph. The PET detectors based on scintillator strips form a shape of a barrel inside the main MRI magnet and a whole-body transceiver coil of the MRI system does not influences gamma quanta propagation from the sample to the PET detectors. In spite of a possibility of the whole body and simultaneous PET and MR imaging still remains the problem of the dedicated MRI coils use. According to the disclosure, such a dedicated coil can be only placed inside the volume closed by the strip PET detectors, so that the dedicated coil elements are on the gamma quanta propagation path. Moreover, the photoelectric converters are placed outside the PET/MRI scanner magnetic field, so long plastic scintillators or optical fibers are required for scintillating light transfer to the converters. Longer optical path of course weakens the photon flux reaching the converters and lowers the PET detection efficiency.

U.S. Pat. No. 8,706,189 B2 disclosed a combined PET/MRI device along with such component as a local coil which special design limits its negative influence on gamma quanta detection in PET system, being outside the local coil. This tends to improve the situation, however, does not avoid the problem completely and requires several modifications of the standard transceiver coils, both in used construction materials, their mechanical and physical properties but also in the arrangement of coil electronics.

So far discussed disclosures present a hybrid systems where PET detectors are integrated with MRI system into a one single device. This of course excludes a possibility of use an existing MRI scanner to extend its functionality with PET detection system.

U.S. Pat. No. 7,728,590 B2 has disclosed a system being a MRI transceiver coil and PET detection system included in a single device, possibly the dedicated coil as well. The device relies on a MRI coil that is in fact divided into a two wire antennas separated by the PET detector arranged radially. The MRI coil is called there as “semi-bird cage” coil. Popular and the most common bird cage coils realizations are the coils of the cylindrical geometry where two wire antenna loops are placed at the cylinder's opposite bases and are coupled by the longitudinal set of wires along the surface of the cylinder. The solution disclosed in mentioned patent gives however a possibility of performing simultaneous MRI and PET scan techniques, but requires a special MRI reconstruction methods based on the magnetic resonance signals from two independent coils as well as specially designed pulse sequences as the excitation pulse is sent by these two coils to the sample volume. Presented arrangement restricts the coil design to the geometry where the MRI coil is being interleaved by the PET detectors, so many popular in MRI techniques coils realisations may be difficult to adapt to this hybrid MRI and PET concept, such as paired saddle coil, popular for the knee MRI diagnostics and surface coils In both quoted inventions descriptions U.S. Pat. No. 8,706,189 B2 and U.S. Pat. No. 7,728,590 B2 a worsening of the coil parameters in comparison to the standard coils in terms of MRI pulse generation and MRI signal detection efficiency may be expected. Moreover, modification of a coil geometry as well as use of a different materials to its construction, results in a need for dedicated changes in a software and hardware of a MRI system to properly control the MRI pulses sent by the transceiver coil to the imaged object as well as image reconstruction methods from recorded resonance signals. All of these have to be well optimized to ensure a good MRI image quality, that nevertheless, may not equal the quality met in standard MRI coils alone.

Therefore, it would be favourable to construct a device which could be a dedicated diagnostic transmit-receive coil consisting of wire transmit-receive antenna which volume can be optimized to the size of the diagnosed object, with the plastic scintillator detectors integrated in a one device, which would enable to register gamma quanta, and perform the magnetic resonance scan simultaneously. The technical problem faced by this invention is to provide such a dedicated TOF-PET/MRI transmit receive coil, which provides low-noise performance with improved sensitivity. Moreover it is an object of the invention to provide TOF-PET/MRI transmit receive coil composed of wire transmit-receive antenna, capable of registering the MRI signal, with the PET capabilities for the MRI scanners, which means that TOF device would be integrated physically with the transmit-receive coil, which as a one device could be placed in the MRI scanner. In this way it would be possible after placing the hybrid coil in the MRI scanner to perform the simultaneous PET and MRI imaging without mutual negative influence of the PET and MRI systems. Surprisingly, said problems were solved by the present invention.

The subject of the present invention is the dedicated hybrid TOF-PET/MRI transceiver coil, comprising: MRI coils in a form of complex shape wire antennas, electronic circuit module allowing for transmitting MRI radio-frequency pulse, and receiving response in a form of magnetic resonance signal, PET detectors arranged longitudinally, fixed permanently to the MRI coils system mechanical support, an electronic signal processing unit, characterized in that the PET detectors are in a form of plastic scintillating strip modules equipped with photoelectric converter units at both ends to convert a light signals from the scintillating module to electrical signals and composed of non-magnetic materials.

In the preferred embodiment of the present invention the inner surface of the mechanical support of transceiver coil is filled with plastic scintillating strip modules in the way that the plastic scintillating strip modules are parallel to the coil antenna rods. In another preferred embodiment of the invention the MRI coils and PET detectors are placed in one housing. Preferably mutual position of the MRI coils and the PET detectors are fixed, to each other. In the preferred embodiment of the invention MRI coils have a birdcage shape, which is in cylindrical geometry, where two wire antenna loops are placed at the cylinders opposite bases and are coupled by the longitudinal set of coil antenna rods along the surface of the cylinder. In another preferred embodiment of the invention the signals registered by the MRI coil and the signals registered by the PET detectors are readout by the same electronic data acquisition system. Preferably the signal readout from the MRI coils and PET detectors are synchronised by the same triggering system.

In present disclosure, as an example of possible embodiment, the “bird-cage” coil geometry is shown and the specific structural and functional details disclosed herein are merely representative for purposes of describing example embodiments. The present invention, however, may be embodied in many alternate forms and should not be construed as limited to only the example embodiments set forth herein.

The main and most important advantage of the proposed solution is possibility of combining standard, well known and investigated MRI coils constructions with PET detection system. In the worst case only a minor changes in the overall MRI coil dimensions will be needed, that do not influence the coil shape, geometry and material properties and therefore is easy to introduce to an existing MRI hardware and software. Also an MRI images quality will not suffer from both the possible size changes and by the presence of PET detection system. Such a hybrid coil can be used in existing MRI systems provided that a feature of PET detection system control will be added to the system or external dedicated independent computer and electronic unit delivered. PET detection system, included in hybrid PET/MRI coil is adapted with its geometry to the geometry of an existing MRI coil and different MRI coil realizations can be considered without restrictions for “semi-” constructions. The proposed design according to the present invention omits the problem of dividing the MRI transceiver-receive antenna of the coil system into two or more sections. The PET detectors used in the present invention covers the whole inner surface of the coil antenna therefore, the PET signal can be registered from the same volume as the MRI signal. The coil antenna and the PET detectors can be placed near the diagnosed volume therefore what is beneficial in terms of both the excitation pulse production in the volume by the electronics of the MRI system as well as image reconstruction based on a signal received by these coils. Moreover the presence of PET detector is not changing the performance and an efficiency of signal detection of transceiver MRI coil as itself in comparison with the coil alone having the same geometric and electronic properties. In addition it is possible, that the dedicated hybrid TOF-PET/MRI coil could be used in existing MRI systems provided that necessary software update is made in computer for MRI system control or dedicated computer system is introduced to control the PET detector acquisition.

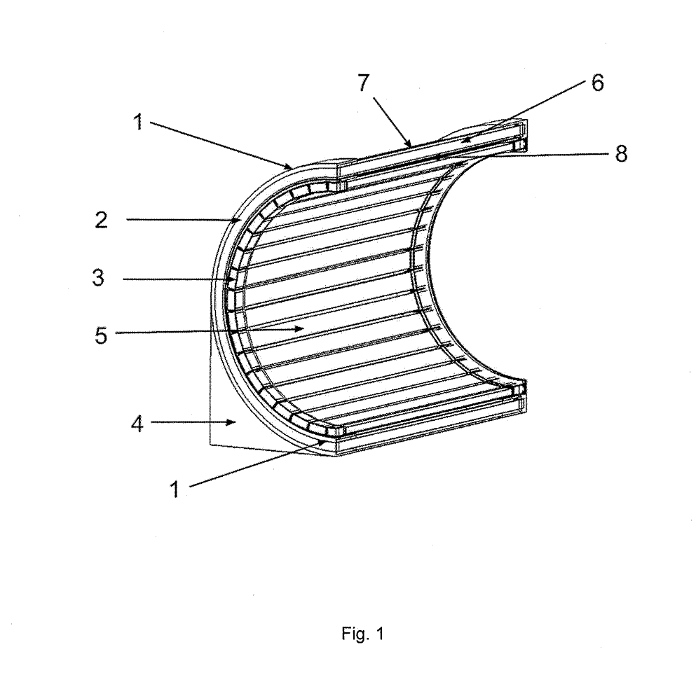

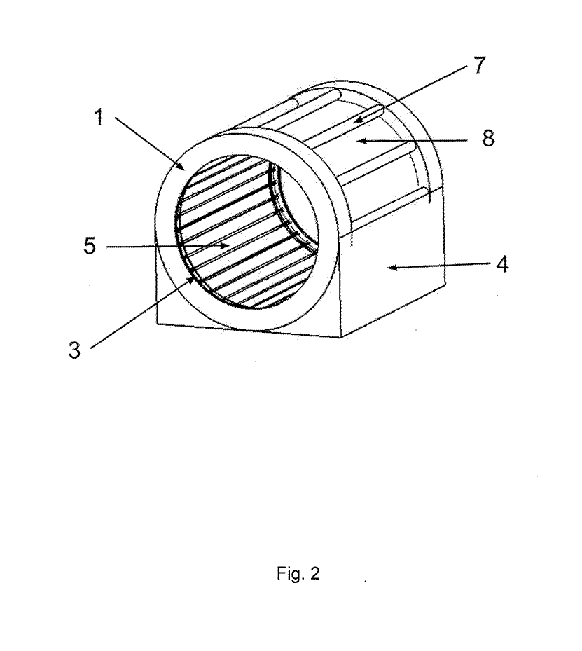

An exemplary embodiment of the present invention is shown in the drawing, in which: FIG. 1 shows isometric view of the cross section of the body dedicated hybrid PET/MRI coil according to the one embodiment of the present invention, and FIG. 2 shows isometric view of the body dedicated hybrid PET/MRI coil according to the one embodiment of the present invention.

### EXAMPLE 1

The dedicated hybrid PET/MRI coil of the present invention is composed of the plastic scintillating strips 5 arranged longitudinally in the form of cylinder, in such a way that the diagnosed object can be placed inside the cylinder parallel the scintillating strips 5. Each single scintillating strip 5 is connected on both ends with photoelectric converter unit 3 in such way that photoelectric converter unit sensitive area is optically connected to the scintillating strip 5. The photoelectric converter units 3 are coupled with the coil housing 1 in such a way that each photoelectric converter unit 3 is fixed. The wire antenna loops 2 are mounted inside the coil housing 1 and coupled through coil antenna rods 6 which are mounted in the wire rod housing 7 in such way that together they are forming a transceiver MRI coil. Between the layer of the scintillating strips 5 and coil 1, 2, 6, 7 mechanical supporting layer 8 is inserted. The coil housing 1 is fixed to mechanical support base 4 in such a way that overall hybrid PET/MRI coil construction is stabilized.

1. A dedicated hybrid TOF-PET/MRI transceiver coil, comprising:
MRI coils in a form of complex shape wire antennas,
electronic circuit module allowing for transmitting MRI radiofrequency pulse, and receiving response in a form of magnetic resonance signal,
PET detectors arranged longitudinally, fixed permanently to the MRI coils system mechanical support,
an electronic signal processing unit,
characterized in that
the PET detectors are in a form of plastic scintillating strip modules equipped with photoelectric converter units at both ends to convert light signals from the scintillating module to electrical signals, and composed of non-magnetic materials.

2. The dedicated hybrid TOF-PET/MRI transceiver coil according to claim 1, characterised in that the inner surface of the mechanical support of transceiver coil is filled with plastic scintillating strip modules in the way that the plastic scintillating strip modules are parallel to the coil antenna rods.

3. The dedicated hybrid TOF-PET/MRI transceiver coil according to claim 1, characterised in that the MRI coils and PET detectors are placed in one housing.

4. The dedicated hybrid TOF-PET/MRI transceiver coil according to claim 1, characterised in that mutual position of the MRI coils and the PET detectors are fixed, to each other.

5. The dedicated hybrid TOF-PET/MRI transceiver coil according to claim 1, characterised in that MRI coils have a birdcage shape, which is in cylindrical geometry, where two wire antenna loops are placed at the cylinders opposite bases and are coupled by the longitudinal set of coil antenna rods along the surface of the cylinder.

6. The dedicated hybrid TOF-PET/MRI transceiver coil according to claim 1, characterised in that the signals registered by the MRI coil and the signals registered by the PET detectors are readout by the same electronic data acquisition system.

7. The dedicated hybrid TOF-PET/MRI transceiver coil according to claim 6, characterised in that the signal readout from the MRI coils and PET detectors are synchronised by the same triggering system.
